(19) United States
(12) Patent Application Publication
(10) Pub. No.: US 2025/0304953 A1
(43) Pub. Date: Oct. 2, 2025

(54) METHOD FOR PURIFYING NUCLEIC ACID LIBRARY

(71) Applicant: SEOUL NATIONAL UNIVERSITY R&DB FOUNDATION, Seoul (KR)

(72) Inventors: Sunghoon KWON, Seoul (KR); Jaewon CHOI, Seoul (KR); Yeongjae CHOI, Seoul (KR); Hansol CHOI, Seoul (KR); Amos Chungwon Lee, Seoul (KR); Taehoon RYU, Gwangmyeong-si (KR)

(21) Appl. No.: 18/273,517

(22) PCT Filed: Feb. 15, 2022
(86) PCT No.: PCT/KR2022/002240

§ 371 Date: Jul. 20, 2023

(30) Foreign Application Priority Data
Feb. 18, 2021 (KR) 10-2021-0021723

(51) Int. Cl. C12N 15/10

(52) U.S. Cl. CPC C12N 15/1065; C12N 15/1068

(57) ABSTRACT

Provided are a method for purifying a nucleic acid library, and a kit, the method comprising the steps of: providing a nucleic acid library comprising single-stranded template nucleic acids; obtaining a library of complementary nucleic acids by binding complementary nucleic acid units to each base of the strand of the template nucleic acids; introducing at least one modified nucleic acid unit during the binding process of the nucleic acid units; and selectively selecting a nucleic acid having a desired length from the library of complementary nucleic acids using the modified nucleic acid unit. According to the present invention, the nucleic acid library may be purified regardless of the complexity, sequence or length of the nucleic acid library, and nucleic acids having different lengths may be simultaneously purified. The purification may be carried out through direct experiment or using a next-generation sequencing instrument.

### CROSS-REFERENCE TO RELATED APPLICATIONS

This application is the U.S. National Phase under 35 U.S.C. § 371 of International Application No. PCT/KR2022/002240 filed on Feb. 15, 2022, which in turn claims the benefit of Korean Application No. 10-2021-0021723, filed on Feb. 18, 2021, the disclosures of which are incorporated by reference into the present application.

### SEQUENCE LISTING

A SEQUENCE LISTING is submitted in a file named PUS230054 ST25.txt via Patent Center and is hereby incorporated by reference in its entirety. Said file was created on Jan. 19, 2024, and is 1,828 bytes in size.

### TECHNICAL FIELD

The present invention relates to a technology capable of purifying only error-free nucleic acid molecules from a nucleic acid library, and to a method capable of purifying them with single-base resolution regardless of sequence, length or complexity.

### BACKGROUND ART

Most of the errors that occur when synthesizing nucleic acids chemically may be referred to as length errors, such as deletion or insertion of a part of the sequence. A poly-acrylamide gel electrophoresis (PAGE) purification method and a high-performance liquid chromatography (HPLC) purification method have conventionally been mainly used to purify nucleic acids. Their principle is based on a method of selecting only nucleic acids having an intended length by using the difference in mobility between error-free nucleic acids and nucleic acids with length errors. However, these methods may be applied to the isolation of one type of nucleic acid strand, but have a limitation in that purification efficiency is low when various types of molecules are in a nucleic acid library.

Meanwhile, there is a technique for purifying error-free nucleic acids by utilizing an error-correction enzyme that recognizes errors on nucleic acids. However, there is a limitation in that it is difficult to apply when various types of molecules are in the nucleic acid library. There is a method of analyzing the sequence of nucleic acids through a next-generation sequencing (NGS), and then selectively recovering error-free nucleic acids from nucleic acids present on a substrate used for analysis. However, this method has the disadvantage of low recovery efficiency because one type of nucleic acid confirmed to be error-free must be recovered individually, so that it is difficult to apply to a nucleic acid library.

As described above, among conventional nucleic acid purification methods, there is no technology capable of purifying nucleic acids at high throughput regardless of the complexity of the library, and thus, there is a need for improvement on these.

### DISCLOSURE

### Technical Problem

According to an aspect of the present invention, herein are provided a method for purifying a nucleic acid library, comprising the steps of: providing a nucleic acid library comprising single-stranded template nucleic acids; obtaining a library of complementary nucleic acids by binding complementary nucleic acid units to each base of the strand of the template nucleic acids; introducing at least one modified nucleic acid unit during the binding process of and selectively selecting a nucleic acid having a desired length from the library of complementary nucleic acids using the modified nucleic acid unit.

According to an embodiment, the nucleic acid library may comprise at least one nucleic acid with a length error due to insertion or deletion of bases.

According to an embodiment, the single-stranded template nucleic acids may be attached to a support.

According to an embodiment, the single-stranded template nucleic acids may comprise a primer region and a library information region.

According to an embodiment, the binding cycle of the nucleic acid unit to the template nucleic acid is repeated and one nucleic acid unit is bound during one cycle, and then the complementary nucleic acid chains may be sorted based on their length.

According to an embodiment, the nucleic acid unit or the modified nucleic acid unit may have a terminator moiety.

According to an embodiment, the nucleic acid unit or the modified nucleic acid unit may have a label moiety.

According to an embodiment, the binding cycle of the nucleic acid unit or the modified nucleic acid unit may comprise a process of binding one nucleic acid unit and a process of removing the terminator moiety.

According to an embodiment, the modified nucleic acid unit may comprise a modified site consisting of an organic material or an inorganic material, wherein the modified site may be one or more selected from the group consisting of a functional group, a magnetic material, a label, and a separate nucleic acid chain.

According to an embodiment, a plurality of binding sites of the modified nucleic acid unit may be set to simultaneously purify nucleic acids having different lengths corresponding to the difference in binding sites.

According to an embodiment, the nucleic acid unit may be one type of nucleotide, or degenerate bases in which several types of nucleotides are mixed.

According to an embodiment, the nucleic acid library may comprise a library composed of degenerate sequences.

According to an embodiment, the nucleic acid library may be purified using a next-generation sequencing instrument.

According to another aspect of the present invention, herein is provided a kit for purifying a nucleic acid library, comprising a primer; a nucleic acid unit having a terminator moiety; a modified nucleic acid unit having a terminator moiety; and a nucleic acid polymerase.

According to an embodiment, the kit may comprise one ore more selected from the group consisting of a magnetic complex having a site capable of binding to the modified nucleic acid unit, a magnet for isolating nucleic acid bound to the magnetic complex, and an alkaline solvent capable of converting double-stranded nucleic acids into single-stranded nucleic acids.

### DESCRIPTION OF DRAWINGS

FIG. 1 shows a process flow chart of the method for purifying a nucleic acid library according to an embodiment of the present invention.

FIG. 2 shows a method for purifying a complex oligo library prone to insertions and deletions.

FIG. 3 shows a method for immobilizing nucleic acids on a substrate and purifying them by hands-on, and a method for purifying them using a next-generation sequencing instrument.

FIG. 4 is a process flow chart showing the progress of the nucleic acid length-based purification technology.

FIG. 5 shows primer sequences used in this experiment.

FIG. 6 shows the actual sequence and purification process of nucleic acids having two lengths.

FIG. 7 shows the actual sequence structure and purification process of a library composed of 4,503 kinds of oligos.

FIG. 8 shows the results of purifying the human genomic capture probe library using a NGS instrument.

FIG. 9 shows the results of analyzing the repetitive sequence, GC percentage and minimum free energy (MFE) distribution in oligo library designs used for purification.

FIG. 10 shows the results of purifying a nucleic acid library used in a nucleic acid-based information storage technology according to an embodiment of the present invention.

FIG. 11 shows the actual sequence structure and purification process of the oligo library in which digital information is stored.

FIG. 12 shows the number of different sequences that may theoretically be present in a library in which digital data is stored, among libraries used for purification.

FIG. 13 shows the result of analyzing the length distribution before and after purification of a library in which digital information is stored. It can be seen that the designed length is 45 bp, and the percentage of length error-free oligos after purification increased from 83% to 97%.

FIG. 14 shows the result of analyzing the percentage of error-free DNA before and after purification of a library in which digital information is stored. It can be seen that the percentage of error-free DNA was analyzed for each address, and in particular, the percentage of error-free DNA of the oligo having an address value of 35 was significantly improved.

FIG. 15 shows a codon table used to encode digital information. Degenerate bases W and S were utilized to maintain high diversity of the oligo library.

FIG. 16 shows the results of purifying an artificial antibody library.

FIG. 17 shows the results of analyzing how many different molecules are present in the library before and after purification.

FIG. 18 shows the actual sequence structure and purification process of the oligo library in which artificial antibody sequence information is stored.

FIG. 19 shows degenerate codon information used in the process of designing an artificial antibody sequence.

### BEST MODEL

Hereinafter, preferred embodiments of the present invention will be described in detail. In describing the present invention, if it is judged that the specific description of the related known technologies may obscure the gist of the present invention, the detailed description thereof will be omitted.

Since various modifications may be made to the present invention and the present invention may have various embodiments, specific embodiments will be illustrated and described in detail in the detailed description. However, this is not intended to limit the present invention to specific embodiments, it is to be understood that this includes all modifications, equivalents, and substitutes included in the spirit and technical scope of the present invention.

The terms used in the present specification are for the purpose of describing specific embodiments only and are not intended to limit the present invention. Singular expressions include plural expressions unless the context clearly indicates otherwise. In the present invention, terms such as “comprise,” “have,” and the like are intended to indicate that there is a feature, number, step, operation, component, part, or combination thereof described in the specification, and it should be understood that the terms do not exclude in advance the possibility of the presence or addition of one or more other features, numbers, steps, operations, components, parts, or combinations thereof.

As used herein, the terms “nucleic acid,” “polynucleotide” and “oligonucleotide” refer to polymers of deoxyribonucleotides or ribonucleotides, either in linear or circular arrangement, and in single- or double-stranded form. These terms are not to be construed as limiting with respect to the length of the polymers. The terms may include known analogues of natural nucleotides as well as nucleotides modified from base, sugar and/or phosphate moieties (for example, phosphorothioate backbones). Generally, and unless otherwise specified, analogs of a specific nucleotide have the same base pairing specificity, that is, an analog of A will be a base pair with T. The term “nucleic acid” is a term in the art that refers to a series of at least two base-sugar-phosphate monomeric units. Nucleotide is a monomeric unit of nucleic acid polymers. The term includes deoxyribonucleic acid (DNA) and ribonucleic acid (RNA) in the form of messenger RNA, antisense, plasmid DNA, parts of plasmid DNA, or genetic material derived from viruses. Antisense is a polynucleotide that interferes with DNA and/or RNA function. Natural nucleic acids have phosphate backbones, and artificial nucleic acids may include different types of backbones, but includes the same bases. The term also includes peptide nucleic acids (PNAs), phosphorothioates, and other variants of the phosphate backbone of natural nucleic acids.

Hereinafter, the present invention will be described in detail through drawings. The method for purifying a nucleic acid library according to an aspect of the present invention relates to a method capable of removing nucleic acids with a length error (insertion or deletion) with single-base resolution.

The technical principle of the method for preparing a nucleic acid library according to the present invention is as follows. The nucleic acid to be purified is preferably isolated into single-stranded nucleic acid for purification. Thereafter, a primer is bound, and N nucleotides are bound. A nucleotide having a terminator moiety are used to bind N nucleotides, and in this process, a next-generation sequencing instrument may be used. This is possible because sequencing by synthesis (SBS), which is a principle of next-generation sequencing, applies a nucleotide having a terminator.

A polymerase is used to link nucleotides. Chemical modification may include linking a biomolecule such as biotin to a nucleotide, adding a functional group such as a thiol group or an amine group thereto, or any click chemistry including these. Error-free nucleic acids may be purified, by applying the fact that after N nucleotides are bound, nucleotides with chemical modifications bind only to error-free nucleic acids. In this case, various bond separation methods may be used according to the chemical modification using avidin family proteins or compounds such as maleimide or N-hydroxysuccinimide ester reactive group. Since this method is a way of recognizing and purifying the type of nucleotide Nth away from the bound primer, nucleic acid libraries having different designed lengths may be simultaneously purified regardless of the sequence, complexity and length of the nucleic acids.

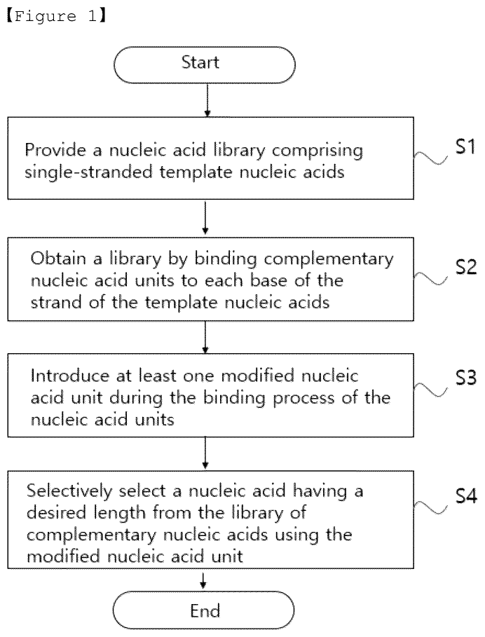

FIG. 1 shows a process flow chart of the method for purifying a nucleic acid library according to an embodiment of the present invention. Referring to FIG. 1, the method comprises the steps of: providing a nucleic acid library comprising single-stranded template nucleic acids (S1); obtaining a library of complementary nucleic acids by binding complementary nucleic acid units to each base of the strand of the template nucleic acids (S2); introducing at least one modified nucleic acid unit during the binding process of the nucleic acid units (S3); and selectively selecting a nucleic acid having a desired length from the library of complementary nucleic acids using the modified nucleic acid unit (S4).

The purification method may be carried out through direct experiment or using a next-generation sequencing instrument. The steps of each process are described in more detail as follows.

In step S1, first, a nucleic acid library comprising single-stranded template nucleic acids to be purified is prepared. The nucleic acids may include deoxyribonucleic acids (DNAS), ribonucleic acids (RNAs), peptide nucleic acids (PNAs), locked nucleic acids (LNAs), glycol nucleic acids (GNAs), threose nucleic acids (TNAs), xeno nucleic acids (XNAs), hexitol nucleic acids (HNAS), synthetic nucleic acids, modified nucleic acids, morpholinos, or combinations thereof. The template nucleic acid chain may include all types of nucleic acids whose sequences are to be known through analysis, and may include genomic DNA, plasmids, oligonucleotides, and the like.

According to an embodiment, in order to increase the diversity of products in the purification process, the template nucleic acids may be designed to bind to any nucleic acid unit. In this case, the sequence of products may change depending on the nucleic acid to be added. Preferably, the template nucleic acids may be composed of a universal base. The universal base is a base containing 3-nitropyrrole, and is a base that may bind to all kinds of bases through a stacking interaction.

In this case, the nucleic acid library is preferably used in a single stranded form for purification. If the target library consists of double-stranded nucleic acids, alkaline solvents such as NaOH may be used to convert the double-stranded nucleic acids into single-stranded nucleic acids. The nucleic acid library may be a library for gene synthesis, an artificial antibody sequence library, a library in which digital information is encoded, a nucleic acid-based vaccine/therapeutic agent library, or a library for nanostructure synthesis, and may preferably be a nucleic acid library obtained by microarray-based synthesis technology in that millions of nucleic acids may be simultaneously synthesized. The nucleic acid library may be provided in the form of a solution or lyophilized powder.

The nucleic acid may be separated from a double-stranded nucleic acid or may be synthesized as a single-stranded nucleic acid from the beginning. Preferably, the nucleic acid library may be a synthesized oligonucleotide, which is a nucleic acid of several to hundreds of nucleotide units, typically 100 to 200 bases.

The nucleic acid library may include at least one nucleic acid with a length error due to insertion or deletion of bases.

The single-stranded template nucleic acids used for purification may be attached to a support. Due to the support, molecules other than the fixed template nucleic acids may be removed, and it serves to allow N nucleotides to link. The support may be a microparticle, a hydrogel, or a solid substrate. The microparticle may have the shape of a bead, rod, disk, plate, or the like, and in some embodiments, preferably, the support may include a magnetic material for biotin-streptavidin reaction and selective isolation of error-free nucleic acids. The solid substrate may be a slide glass, a microarray substrate, a hydrogel, a polymer, a microparticle, or the like. In order to attach the template nucleic acids to the support, the support or the template nucleic acids may each be modified with a reactive group. For example, the support may be coated with an N-hydroxysuccinimide (NHS) ester group, and the template nucleic acids may be modified with an amine group.

For polymerization, forward and reverse primers for amplification may be coupled to the single-stranded template nucleic acids. As a result, the single-stranded template nucleic acids may include a primer region and a library information region.

The method for purifying a nucleic acid library according to the present invention may be applied to all nucleic acids library regardless of the sequence, complexity or length of the nucleic acid library.

In step S2, a library of complementary nucleic acids is obtained by binding complementary nucleic acid units to each base of the strand of the template nucleic acids. The nucleic acid units may be one or more selected from the group consisting of nucleotides, nucleosides, oligonucleotides and polynucleotides. A polymerase may be used to bind the nucleic acid units.

Preferably, in the process of obtaining a library of complementary nucleic acids, the nucleic acid units may be repeatedly bound together with the binding of a primer, for example, a length error-free nucleic acid sequence having N bases may be obtained by binding N nucleotides.

In this case, preferably, a nucleic acid unit having a function of a reversible terminator may be used as the nucleic acid unit. In order to serve as the reversible terminator, the nucleic acid unit may have a blocking group capable of reversible attachment and detachment after binding to the template nucleic acid, i.e., a terminator moiety, and may further have a label moiety (for example, fluorophore) for sequence identification.

The function as a reversible terminator may be achieved by controlling the insertion of monomers by attaching and detaching a blocking group, and by the process of recognizing a base type.

In order to obtain a library of the complementary nucleic acids, a sequencing-by-synthesis (SBS) method used in next-generation sequencing analysis may be applied. SBS uses a fluorescently labeled nucleotide monomer, and is a technology that by inserting each monomer by the polymerase and then detecting the fluorescent signal labeled on the monomer, allows the base of the inserted monomer to be recognized and at the same time the complementary base to be analyzed. The nucleoside triphosphate (dNTP) used in the SBS technology is generally in the form of a blocking group (3′-O-blocking group) from which the 3′-OH portion and the base portion may be each reversibly removed, and a dual-modified reversible terminator (DRT) labeled with a fluorophore. In this case, each of the four bases (A, T, G, C) is labeled with a different fluorescent fluorophore. When the polymerization with these monomers is performed using the DNA to be analyzed as a template chain, the monomer is inserted by DNA polymerase, and then the next monomer is not inserted because the 3′-OH is blocked by the blocking group, and as a result, the polymerization reaction is temporarily stopped. In this case, the type of the inserted base may be known through the detection of the fluorescence of the fluorophore labeled on the base portion of the inserted monomer, so that the complementary base sequence in the template chain may be analyzed. Since the 3′-OH functional group is restored when the fluorescent group and the 3′-O-blocking group are removed, a monomer in the next sequence may be inserted, and the base of the template chain may be analyzed by recognizing the base type of the monomer inserted in the same way. Sequencing-by-synthesis (SBS) is a technique of sequentially synthesizing and analyzing sequences while repeating this process.

In order to obtain the library of the complementary nucleic acids, nucleoside, nucleotide (nucleoside monophosphate), nucleoside diphosphate, nucleoside triphosphate, or the like may be used as the nucleic acid unit. In terms of binding efficiency, the nucleic acid unit may preferably be a nucleoside triphosphate such as ATP, GTP, CTP, TTP, UTP, ITP, XTP, dATP, dGTP, dCTP or dTTP.

The nucleobase in the nucleic acid unit may be a purine base (adenine, guanine, hypoxanthine, xanthine, purine analog) or a pyrimidine base (uracil, thymine, cytosine, pyrimidine analog). Types of the base may include both natural bases such as adenine, guanine, thymine (uracil) and cytosine, and non-natural bases.

The nucleotide or nucleoside portion in the nucleic acid unit may be chemically modified for high stability or compatibility with various solvents, and for example, the modified nucleic acid unit may include a modified base, including a phosphorothioate, methylphosphonate, peptide nucleic acid, 2′-O-methyl, fluoro- or carbon, methylene or locked nucleic acid (LNA) molecule.

During the polymerization reaction, the nucleic acid unit acts as a reversible terminator, so that one nucleic acid unit may be bound during one cycle in the process of binding the nucleic acid units. By repeating each cycle using this, the intended number of nucleic acid units may be sequentially bound. In this case, if there is no length error in the nucleic acid, the base type (for example, A, G, T, C) of the nucleic acid unit to be bound next may be predicted. If there is a length error in the nucleic acid, the type of base to be bound will change.

In this way, the complementary nucleic acid chains may be sorted based on their length by repeating the binding cycle of the nucleic acid unit to the template nucleic acid and binding one nucleic acid unit during one cycle. The binding cycle of the nucleic acid unit may include a process of binding one nucleic acid unit into which a blocking group has been introduced and a process of removing the blocking group before introduction of a nucleic acid unit in the next sequence.

In an embodiment, the nucleic acid unit may be one type of nucleotide, or degenerate bases in which several types of nucleotides are mixed. The degenerate bases have the advantage of increasing the diversity of a library or increasing the diversity of expressed proteins or phenotypes.

In addition, the nucleic acid library may comprise a library composed of degenerate sequences. By doing so, the synthesis cost for storing unit information may be reduced. In step S3, at least one modified nucleic acid unit is introduced during the binding process of the nucleic acid units. The modified nucleic acid unit may be one in which a modified site in the form of an organic material or an inorganic material is introduced into the nucleic acid unit to capture or isolate the desired complementary nucleic acid chain. For example, the modified site may include a functional group, a magnetic material, a label (fluorophore, barcode, and the like), a separate nucleic acid chain, and the like.

For example, the introduction of a functional group is one of the chemical modifications, and may be performed by a way of linking a nucleic acid unit with a biomolecule such as biotin, a thiol group, an amine group, a phosphate group, other substances used in click chemistry, or the like to a nucleic acid unit.

According to an embodiment, the nucleic acid unit or the modified nucleic acid unit may be composed of one or two or more nucleotides, and preferably may be a trimer capable of encoding one amino acid.

Error-free nucleic acids may be purified by applying the fact that the modified nucleic acid unit binds only to error-free nucleic acids, and the reason for this is that, in the case of nucleic acids with errors, the type of sequence in the next sequence after binding of a series of nucleic acid units is different from the original sequence, and thus, it does not grow into a chain of the same length.

In an embodiment, a plurality of binding sites of the modified nucleic acid unit may be set to simultaneously purify nucleic acids having different lengths corresponding to the difference in binding sites.

The binding site of the modified nucleic acid unit may be determined from the library design stage through the location and base type. If the binding site of the modified nucleic acid unit is designated in advance, it may increase purification efficiency, and it is easy to use in amplification, sequencing and the like of error-free nucleic acids after purification.

In step S4, a nucleic acid having a desired length is selectively selected from the library of complementary nucleic acids using the modified nucleic acid unit. That is, by introducing a modified site for capture or isolation into the complementary nucleic acid, only error-free nucleic acid may be purified using the modified site.

A functional group, a magnetic material, a label, a separate nucleic acid chain and the like included in the modified region may be used to capture or isolate the modified nucleic acid chain by processing an external functional group capable of chemical or physical bonding, an external magnetic force, laser application according to the location information of the label, nucleic acids capable of complementary binding and the like, respectively.

For example, after N−1 nucleic acid units are bound to a template nucleic acid having N sequences, only one type of modified nucleic acid unit complementary to the Nth base located in the next sequence is introduced. In this case, the modified nucleic acid unit has a modified site together with a blocking group serving as a terminator, and as a result, when the modified site introduced into a complementary nucleic acid chain is a chemically modified site, a nucleic acid chain having a desired length may be isolated by various purification methods using avidin family proteins or compounds such as maleimide or N-hydroxysuccinimide ester reactive group. When the modified nucleic acid unit is a biotin-bound dNTP, a desired nucleic acid library may be purified by reacting it with streptavidin-coated magnetic beads capable of forming a complex with biotin and then using magnetic force.

Since the above-described method is a way of recognizing and purifying the type of nucleotide Nth away from the bound primer, nucleic acid libraries having different designed lengths may be simultaneously purified regardless of the sequence, complexity and length of the nucleic acids.

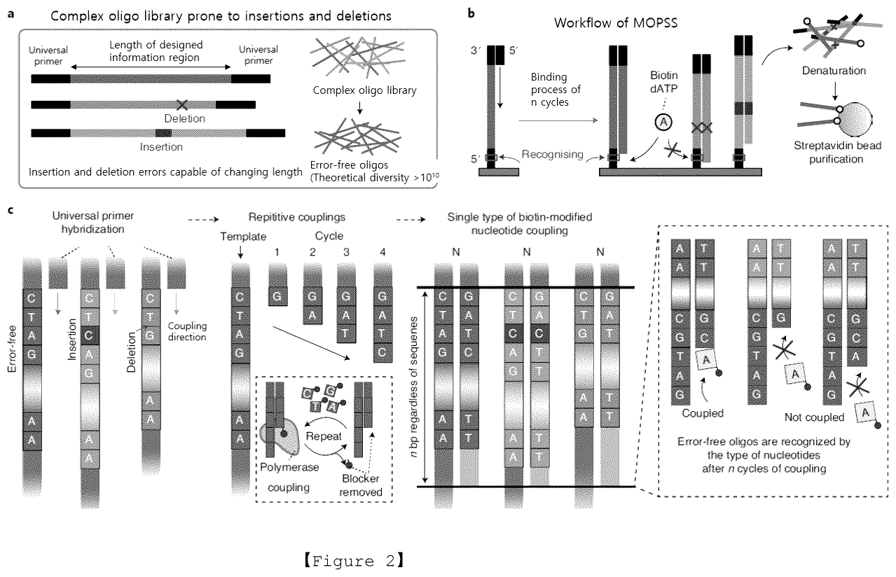

The principle of the method for purifying a nucleic acid library according to the present invention will be described with specific examples. FIG. 2 shows a method for purifying a complex oligo library prone to insertions and deletions. Referring to FIG. 2, the structure of a nucleic acid is composed of a universal primer region and a library information region (a in FIG. 2). The synthesized nucleic acid library is transferred to a solid substrate for purification. After primers are bound to all nucleic acids constituting the library, N nucleotides are bound. In this case, nucleotides with a blocking group and biotin may bind only to error-free nucleic acids at the binding site, but cannot bind to nucleic acids with errors. Thereafter, only error-free nucleic acids are purified with streptavidin-coated magnetic beads (b in FIG. 2). In each cycle, only one nucleotide may be bound due to the blocking group. Thereafter, the blocking group is removed so that the next nucleotide may be bound (c in FIG. 2).

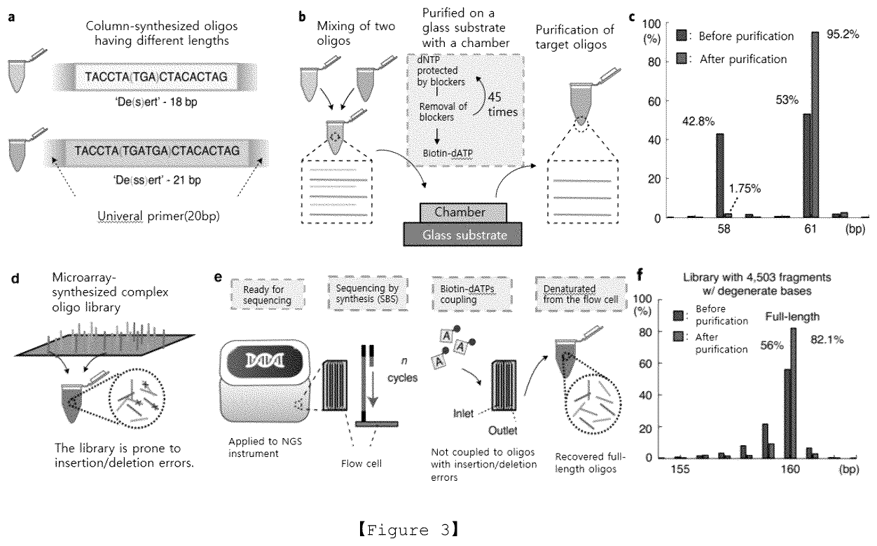

FIG. 3 shows a method for immobilizing nucleic acids on a substrate and purifying them by hands-on, and a method for purifying them using a next-generation sequencing instrument.

a to c in FIG. 3 show the processes and results of immobilizing nucleic acids on a substrate and purifying them by hands-on. A library for purifying long-length nucleic acids was designed by mixing nucleic acids having two different lengths (a in FIG. 3). Column-synthesized oligos having different lengths were composed of a primer region and a library information region of 18 bp (base-pair) and 21 bp, respectively. Long-length nucleic acids were purified by mixing nucleic acids having two different lengths at the same concentration (b in FIG. 3). As a result of purification, the purity of the long-length nucleic acids increased from 53% to 95.2% after purification (c in FIG. 3).

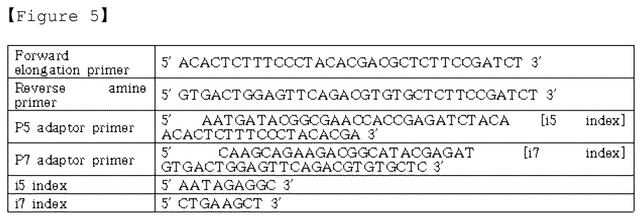

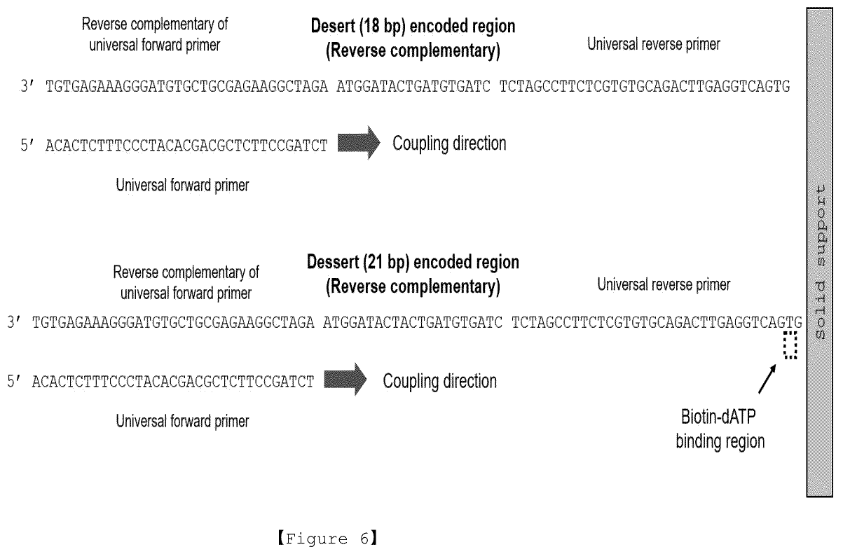

FIG. 5 shows primer sequences used in this experiment. Primers with identical sequences were used in all experiments in subsequent figures. However, even if the sequence of the primer is changed, the purification efficiency is not affected. FIG. 6 shows the actual sequence and purification process of nucleic acids having two lengths. It can be seen that the region for binding of biotin-dATP is marked.

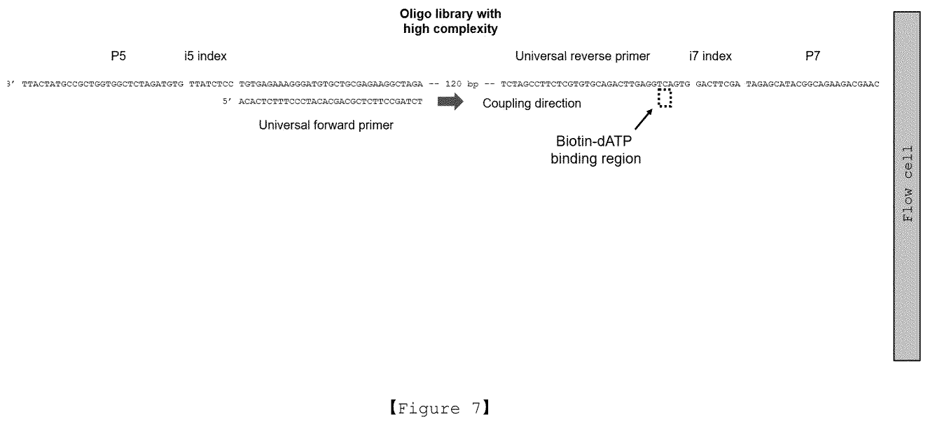

d to f in FIG. 3 show the process and results of purifying the library by an automated method using a next-generation sequencing instrument. By analyzing numerous nucleic acids at high speed using a next-generation sequencing instrument in purifying a nucleic acid library, time and cost may be drastically reduced. A library composed of 4,503 different nucleic acids was purified using an NGS instrument (d in FIG. 3). N nucleotides were bound in the nucleic acid library on an NGS instrument, and nucleotides with terminator and biotin were applied, and then only error-free nucleic acids were purified (e in FIG. 3). The percentage of error-free nucleic acids increased from 56% to 82.1% after purification (f in FIG. 3). FIG. 7 shows the actual sequence structure and purification process of a library composed of 4,503 kinds of oligos. It can be seen that the region for binding of biotin-dATP is marked.

According to another aspect of the present invention, the present invention provides a kit for purifying a nucleic acid. Using the kit, it is possible to conveniently synthesize nucleic acids complementary to a nucleic acid library and select only error-free nucleic acids from them. The kit comprises a primer capable of complementarily binding to a nucleic acid, a nucleic acid unit having a terminator moiety, a modified nucleic acid unit having a terminator moiety, and a nucleic acid polymerase.

The kit may be used in the purification method described above. Therefore, preferably, the kit may comprise one ore more selected from the group consisting of a magnetic complex having a site capable of binding to the modified nucleic acid unit, a magnet for isolating nucleic acid bound to the magnetic complex, and an alkaline solvent capable of converting double-stranded nucleic acids into single-stranded nucleic acids.

The method for purifying a nucleic acid library according to the present invention may be applied to various fields. Nucleic acids are essential materials for a variety of applications, such as synthetic biology, synthetic pharmaceutical engineering, DNA nanotechnology and nucleic acid-based data storage. If the present method is applied, a nucleic acid library may be purified regardless of its diversity, and the purified nucleic acid library may be applied to gene assembly, synthetic antibody screening, and genetic perturbation screening. In addition, the present technology may be applied to the synthesis of various proteins other than antibodies and may also be applied to the field of gene therapy using gene-editing technology using CRISPR found in prokaryotes. Since the present technology is a nucleic acid purification technology and may be applied not only to DNA but also to RNA, it may be applied to RNA interference (RNAi) therapeutic agent, which regulates protein expression by binding to mRNA in a cell, a nucleic acid vaccine, which induces an immune response by injecting a nucleic acid capable of synthesizing an antigen protein into a cell, or the like. The present invention may also be applied to nucleic acid origami, which makes a structure by binding nucleic acids to complementary short staple nucleic acids, and nucleic acid brick technology, which makes a structure by linking short nucleic acids. It may also be applied to manufacturing actuators such as optical sensors, pH sensors and temperature sensors, implementing artificial cell organelles, or manufacturing drug delivery systems that deliver drugs to a desired location, with structures composed of nucleic acids. It may also be applied to constructing nucleic acid probes that detect antibodies, RNAs or proteins to diagnose diseases. The present invention may also be applied to manufacturing and screening aptamers, which are nucleic acids that bind to specific proteins, and may also be applied to computing techniques that constitute a nucleic acid circuit using the presence or absence of complementary binding and the property of nucleic acids to bind to more stable strands. It may also be applied to imaging to reveal the structure of cells or tissues and the spatial location of proteins and nucleic acids by injecting antibodies with aptamers or nucleic acids linked to fluorescent molecules that bind to proteins, RNA or DNA in cells, or by injecting nucleic acids complementary to RNA and DNA, or techniques for quantifying the amount of proteins and nucleic acids.

Hereinafter, the present invention will be described in more detail through examples.

### EXAMPLES

### Example 1: Length-Based Purification Process of Nucleic Acid Library

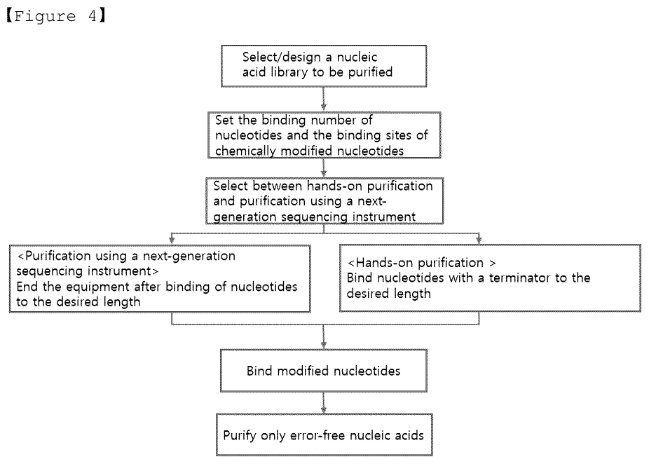

FIG. 4 is a process flow chart showing the progress of the nucleic acid length-based purification technology. The entire experimental process proceeds with immobilization of a nucleic acid library on a support, length-based sorting, a process of binding biotin-bound deoxyadenosine triphosphate (dATP) only to nucleic acids having an intended length, and a process of selecting only biotin-linked nucleic acids having an intended length.

In the examples, the purification process was carried out in two ways: hands-on or automated processes.

### (1) Hands-on Experiment

Nucleic acids were purified hands-on using a substrate on which nucleic acids may be immobilized. Immediately after the nucleic acids were immobilized on a glass substrate, binding of primers and binding of N nucleotides having a protecting group (blocker) and functioning as a reversible terminator proceeded. 3′-O-azidomethyl-dNTPs were used as reversible terminator nucleotides, and tris(2-carboxyethyl)phosphine (TCEP) was used to remove the blocking group. After binding of the Nth nucleotide, biotin-bound 3′-O-azidomethyl-dATPs were used. Biotin-bound error-free nucleic acids were purified using streptavidin-coated magnetic beads.

### (2) Automated Purification Process Using Next-Generation Sequencing Instrument

MiSeq, which is Illumina's next-generation sequencing instrument, was used for purification. The process of binding nucleotides having N terminators was carried out through sequencing by synthesis (SBS) of the instrument, and the rest of the process was carried out in the same way as the previous hands-on purification process.

### 1.1. Immobilization and Length-Based Sorting of a Nucleic Acid Library on a Support

The nucleic acid library used for purification was immobilized on a support in a double-stranded form. 0.1 N NaOH was used to make it single-stranded, and in this case, the 5′ end was immobilized on the support. After binding of the primer, the intended number of nucleotides were allowed to be bound. In this case, using a terminator nucleotide, only one nucleotide was allowed to be bound during one cycle, and it was repeated 45 times for the purification of the nucleic acid library in a to c of FIGS. 3, and 148 times for the purification of the nucleic acid library in d to f in FIG. 3. If there is no length error in the nucleic acid, nucleotides are bound to the intended position, and the next possible nucleotide to be bound may be predicted, and in this example, deoxyadenosine triphosphate was allowed to be bound. In the case of nucleic acids with length errors, the deoxyadenosine triphosphate does not reach or goes beyond the binding position.

### 1.2. Process of Binding Biotin-Bound Deoxyadenosine Triphosphate to Nucleic Acids Having an Intended Length

Deoxyadenosine triphosphate may bind only to length error-free nucleic acids, and biotin-bound deoxyadenosine triphosphate was added to be bound.

### 1.3. Process of Selecting Only Nucleic Acids Having an Intended Length Using Biotin

Biotin is bound only to the 3′ end of the length error-free nucleic acids. Only length error-free nucleic acids were selected at once using biotin-streptavidin interaction with streptavidin magnetic beads. In order to verify the length and error rate of the selected nucleic acids, they were amplified using polymerase chain reaction (PCR) and analyzed using next-generation sequencing (NGS) method.

### Example 2. Automated Purification Process Using a Next-Generation Sequencing Analyzer

Process 1.1 of Example 1 was automated using a next-generation sequencing analyzer. Illumina's MiSeq instrument was used, and the number of repeated binding of nucleotides with terminators was adjusted by adjusting the number of sequencing cycles of the instrument. Thereafter, the process of binding biotin-bound deoxyadenosine triphosphate and the process of selecting only nucleic acids having an intended length were performed in the same manner as in Example 1.

### Example 3. Application of Purification Technology to a Human Genomic Gene Capture Probe Library

The length-based purification technology of a nucleic acid library was applied to a human genomic gene capture probe library. A capture probe library capable of binding to 4,493 genes related to genetic diseases among human genes was synthesized, and length error-free nucleic acids were purified from the library. The library is composed of 11,263 probes of 120 bp, and purification was performed using the next-generation sequencing analyzer specified in Example 2.

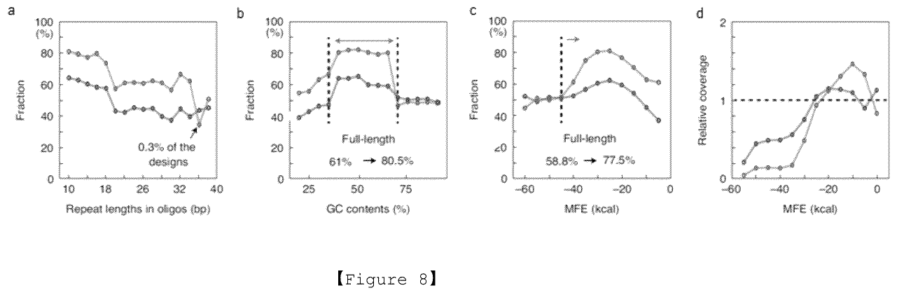

FIG. 8 shows the results of purifying the human genomic capture probe library using a NGS instrument. a in FIG. 8 shows the purification result according to the length of microsatellites or repetitive sequences. b in FIG. 8 shows the purification result according to the percentage of guanine and cytosine bases (GC content), and for oligos with percentages of guanine and cytosine bases between 35% and 70%, the percentage of length error-free nucleic acids improved from 61% to 80.5%. c in FIG. 8 shows the purification result according to the minimum free energy (MFE). For oligos with minimum free energy of −45 kcal/mol or more, the percentage of length error-free oligos improved from 58.8% to 77.5%. d in FIG. 8 shows the relationship with the number of reads in the sequencing result according to the minimum free energy. Despite the progress of purification, relative percentages of sequences in the library did not change significantly.

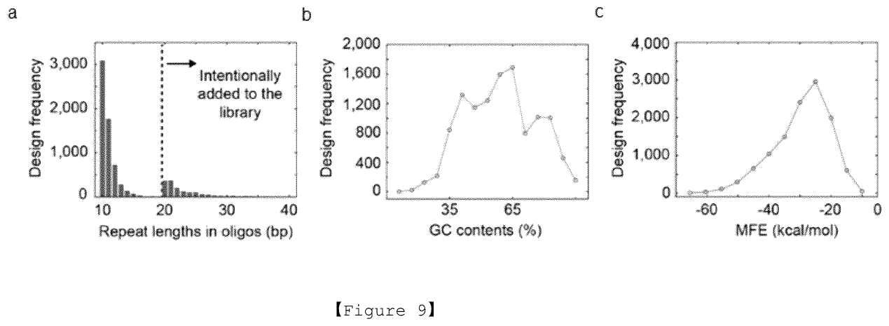

FIG. 9 shows the results of analyzing the repetitive sequence, GC percentage and minimum free energy (MFE) distribution (a to d in FIG. 9, respectively) in oligo library designs used for purification. Designs with repetitive sequence lengths of 20 bp or more were intentionally added to the library. Taken together in FIGS. 8 and 9, it was confirmed that the purification proceeded effectively regardless of repetitive sequences, GC percentages, and MFE values.

### Example 4. Application of Purification Technology to a Nucleic Acid Library in which Digital Data is Stored

The length-based purification technology of a nucleic acid library was applied to a nucleic acid library in which digital data was stored. The nucleic acid library stores 854 bytes of text information and is composed of 45 nucleic acids consisting of 45 bp of degenerate bases.

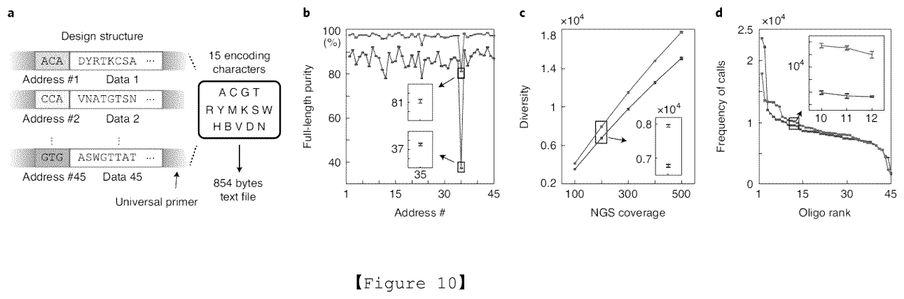

FIG. 10 shows the results of purifying a nucleic acid library used in a nucleic acid-based information storage technology according to an embodiment of the present invention.

According to a in FIG. 10, a nucleic acid library applied to a nucleic acid-based information storage technology is composed of a primer region, an information storage region, and an address region for information sorting. According to b in FIG. 10, the purity of the library was analyzed according to the address, and the purity of the nucleic acid according to each address was sorted in descending order. It can be seen that the purity after purification increases from 83% to 97% on average. c in FIG. 10 is a result showing the diversity of nucleic acids analyzed according to NGS coverage. Considering that the NGS coverage and the diversity of nucleic acids are directly proportional to each other even after purification, it can be seen that purification was successfully performed for a nucleic acid library having a high diversity. d in FIG. 10 shows the result of analyzing the number of times nucleic acids were read for each address when the library was read 400,000 times in the NGS analysis and sorting them in descending order. Considering that the number of times nucleic acids were read for each address before and after purification was similar, it can be seen that the purification process did not affect the distribution of nucleic acids in the nucleic acid library.

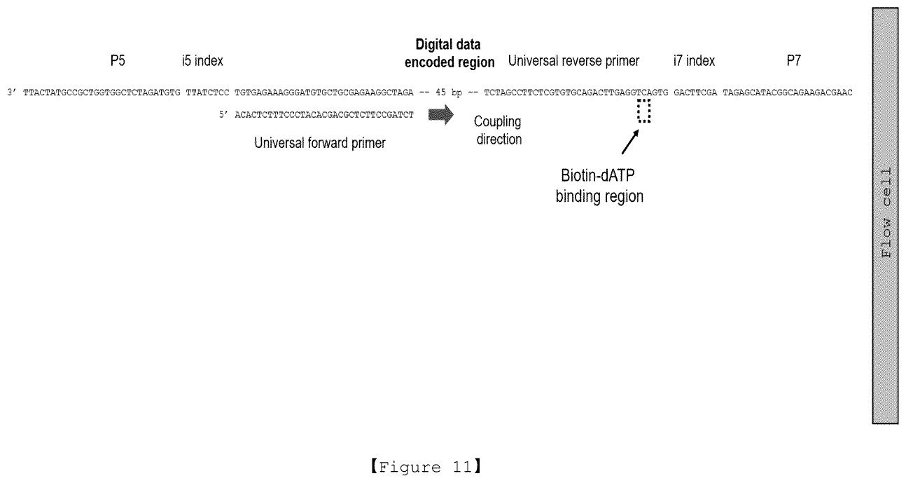

FIG. 11 shows the actual sequence structure and purification process of the oligo library in which digital information is stored. It can be seen that the region for binding of biotin-dATP is marked.

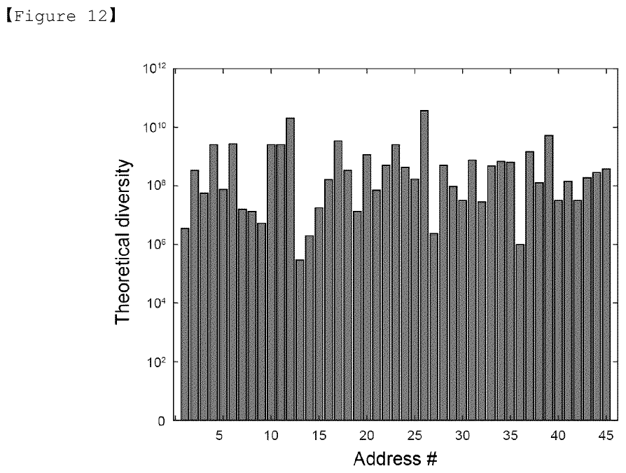

FIG. 12 shows the number of different sequences that may theoretically be present in a library in which digital data is stored, among libraries used for purification. Since different degenerate bases are used for each address, various types of oligos exist.

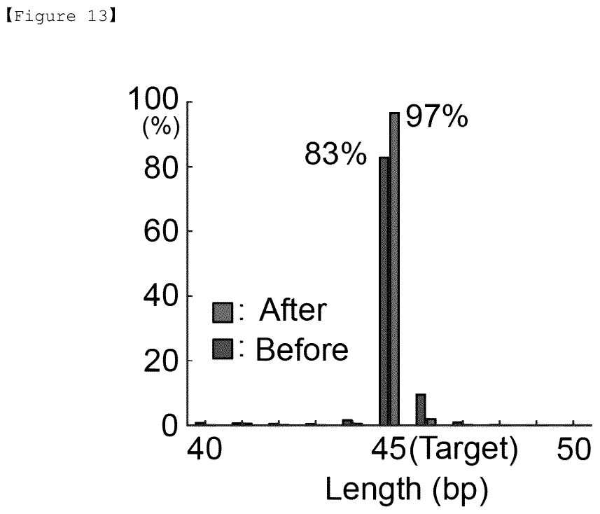

FIG. 13 shows the result of analyzing the length distribution before and after purification of a library in which digital information is stored. It can be seen that the designed length is 45 bp, and the percentage of length error-free oligos after purification increased from 83% to 97%.

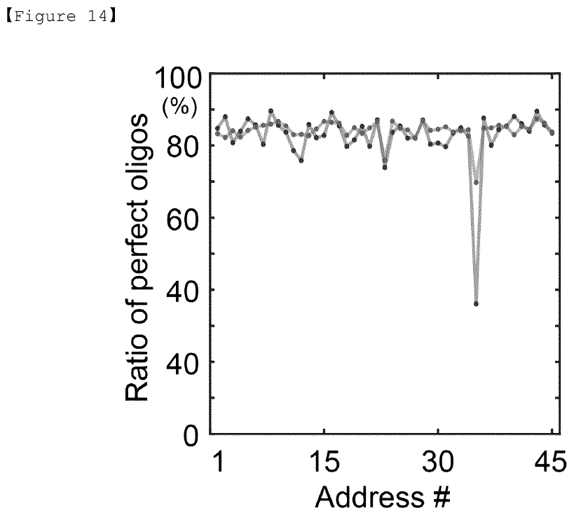

FIG. 14 shows the result of analyzing the percentage of error-free DNA before and after purification of a library in which digital information is stored. It can be seen that the percentage of error-free DNA was analyzed for each address, and in particular, the percentage of error-free DNA of the oligo having an address value of 35 was significantly improved.

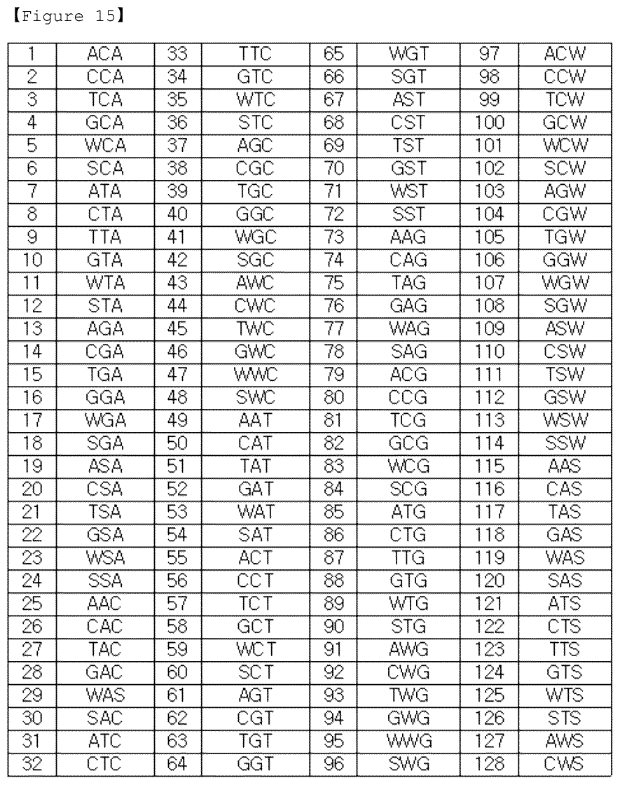

FIG. 15 shows a codon table used to encode digital information. Degenerate bases W and S were utilized to maintain high diversity of the oligo library.

### Example 5. Application of Purification Technology to an Artificial Antibody Nucleic Acid Library

The length-based purification technology of a nucleic acid library was applied to an artificial antibody nucleic acid library. The antibody library encodes the complementarity-determining region (CDR) H3 region, and is composed of CDR H3-1 (112 bp), CDR H3-2 (109 bp), CDR H3-3 (112 bp) and CDR H3-4 (115 bp). Each CDR H3 library was composed of degenerate codons to ensure high diversity.

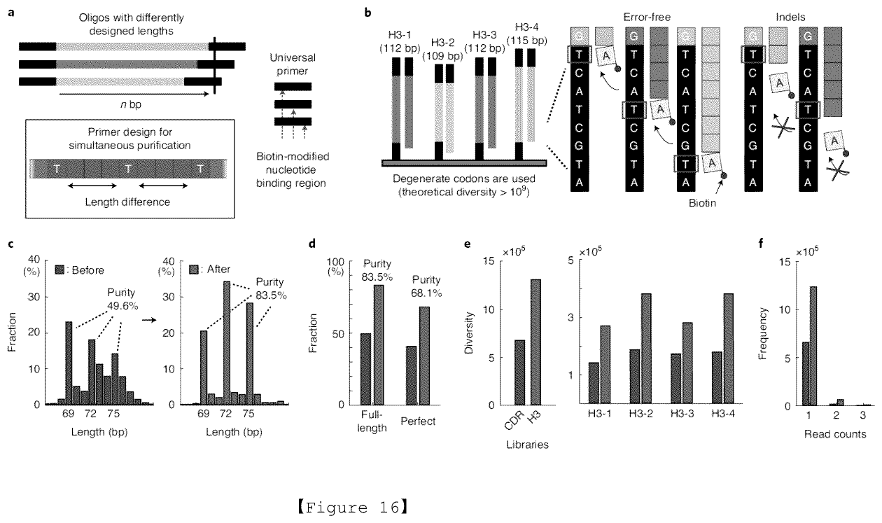

FIG. 16 shows the results of purifying an artificial antibody library. According to a in FIG. 16, it is shown that when a plurality of binding sites of nucleotides with terminator and biotin are intentionally set, nucleic acids having different lengths corresponding to the difference in binding sites may be simultaneously purified. b in FIG. 16 shows a method for simultaneously purifying a plurality of lengths from a nucleic acid library composed of three lengths (109 bp, 112 bp and 115 bp) applied to antibody screening technology. According to c in FIG. 16, the total sum of purity of error-free nucleic acids in each length increased from 49.6% to 83.5% after purification. According to d in FIG. 16, the percentage of in-frame nucleic acids having the correct protein translation framework increased from 36.5% to 80.3% after purification. e in FIG. 16 shows that the percentage of nucleic acids with errors decreased, so that the diversity of in-frame nucleic acids increased after purification.

From the results of FIG. 16, it can be seen that nucleic acids having different lengths may be simultaneously purified with a single-base sequence resolution using a nucleic acid library applied to synthetic antibody screening technology.

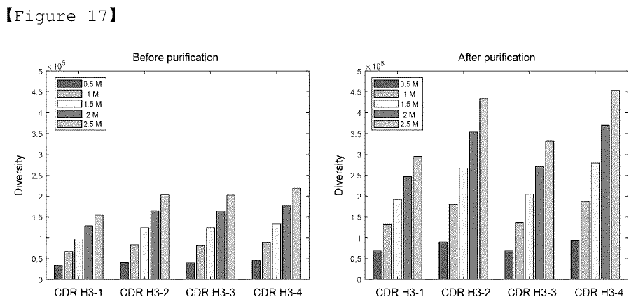

FIG. 17 shows the results of analyzing how many different molecules are present in the library before and after purification. The number of different molecules was analyzed while increasing the number of sequencing reads from 500,000 reads to 2,500,000 reads at intervals of 500,000 reads. Since the complexity of the library is very high, it can be seen that as the number of sequencing reads increases, the number of different molecules also increases linearly. In addition, since the number of error-free oligos decreased after purification, it can be seen that the number of different molecules is greater even if the number of sequencing reads is the same compared to before purification, and the number of different molecules increases linearly as the number of reads increases, as before purification.

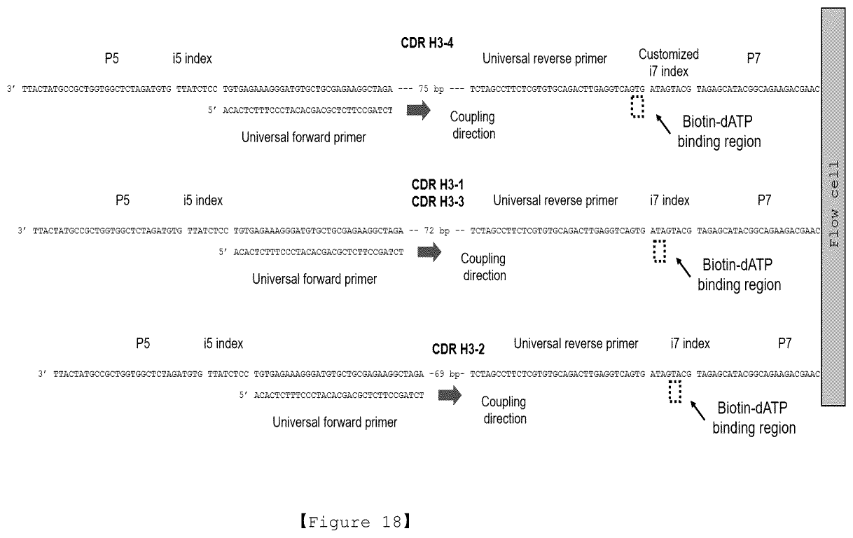

FIG. 18 shows the actual sequence structure and purification process of the oligo library in which artificial antibody sequence information is stored. It can be seen that the artificial antibody sequence is composed of DNA having three different lengths, and the region for binding of biotin-dATP is marked.

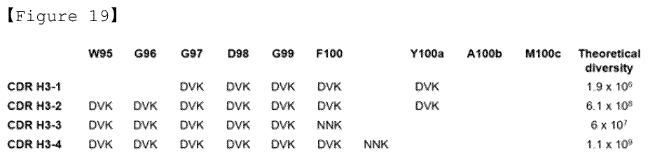

FIG. 19 shows degenerate codon information used in the process of designing an artificial antibody sequence. Different types of degenerate codons were used according to each amino acid position, and the number of theoretically possible different oligos according to the degenerate codon sequence is shown.

### Example 6. Application to Mass Production of DNA/RNA Vaccines And DNA/RNA Therapeutic Agents

The length-based purification technology of a nucleic acid library may be applied to the development f nucleic acid (DNA/RNA)-based vaccines or therapeutic agents. Nucleic acid-based vaccines and therapeutics require a process of culturing E. coli and extracting DNA for a long period of time due to errors in the process of nucleic acid synthesis, resulting in problems of high production costs and low production efficiency. Since the process of culturing E. coli may be omitted by applying nucleic acid purification technology, it may be applied to mass production of nucleic acid-based vaccines or therapeutic agents such as COVID-19 mRNA vaccine.

1. A method for purifying a nucleic acid library, comprising the steps of:
providing a nucleic acid library comprising single-stranded template nucleic acids;
obtaining a library of complementary nucleic acids by binding complementary nucleic acid units to each base of the strand of the template nucleic acids;
introducing at least one modified nucleic acid unit during the binding process of the nucleic acid units; and
selectively selecting a nucleic acid having a desired length from the library of complementary nucleic acids using the modified nucleic acid unit.

2. The method for purifying a nucleic acid library according to claim 1, wherein the nucleic acid library comprises at least one nucleic acid with a length error due to insertion or deletion of bases.

3. The method for purifying a nucleic acid library according to claim 1, wherein the single-stranded template nucleic acids are attached to a support.

4. The method for purifying a nucleic acid library according to claim 1, wherein the step of obtaining a library of complementary nucleic acids comprises binding of a primer and iterative binding of the nucleic acid unit.

5. The method for purifying a nucleic acid library according to claim 1, wherein the single-stranded template nucleic acids comprise a primer region and a library information region.

6. The method for purifying a nucleic acid library according to claim 1, wherein the binding cycle of the nucleic acid unit to the template nucleic acid is repeated, and one nucleic acid unit is bound during one cycle.

7. The method for purifying a nucleic acid library according to claim 6, wherein the nucleic acid unit or the modified nucleic acid unit has a terminator moiety.

8. The method for purifying a nucleic acid library according to claim 7, wherein the nucleic acid unit or the modified nucleic acid unit further has a label moiety.

9. The method for purifying a nucleic acid library according to claim 7, wherein the binding cycle of the nucleic acid unit or the modified nucleic acid unit comprises a process of binding one nucleic acid unit and a process of removing the terminator moiety.

10. The method for purifying a nucleic acid library according to claim 1, wherein the modified nucleic acid unit comprises a modified site consisting of an organic material or an inorganic material.

11. The method for purifying a nucleic acid library according to claim 10, wherein the modified site is one or more selected from the group consisting of a functional group, a magnetic material, a label, and a separate nucleic acid chain.

12. The method for purifying a nucleic acid library according to claim 1, wherein a plurality of binding sites of the modified nucleic acid unit are set to simultaneously purify nucleic acids having different lengths corresponding to the difference in binding sites.

13. The method for purifying a nucleic acid library according to claim 1, wherein the nucleic acid unit is one type of nucleotide, or degenerate bases in which several types of nucleotides are mixed.

14. The method for purifying a nucleic acid library according to claim 1, wherein the nucleic acid library comprises a library composed of degenerate sequences.

15. The method for purifying a nucleic acid library according to claim 1, wherein the nucleic acid library is purified using a next-generation sequencing instrument.

16. The method for purifying a nucleic acid library according to claim 1, comprising the step of designating or designing a position capable of binding to the modified nucleic acid unit in advance with a specific base at a specific position.

17. A kit for purifying a nucleic acid library, comprising a primer; a nucleic acid unit having a terminator moiety; a modified nucleic acid unit having a terminator moiety; and a nucleic acid polymerase.

18. The kit for purifying a nucleic acid library according to claim 17, wherein the kit comprises one ore more selected from the group consisting of a magnetic complex having a site capable of binding to the modified nucleic acid unit, a magnet for isolating nucleic acid bound to the magnetic complex, and an alkaline solvent capable of converting double-stranded nucleic acids into single-stranded nucleic acids.


SEQUENCE LISTING (8 sequences)

SEQ ID NO: 1 - DNA, 18 bp, Artificial Sequence
  Desert (18bp) encoded region
tacctatgactacactag

SEQ ID NO: 2 - DNA, 21 bp, Artificial Sequence
  Dessert (21bp) encoded region
tacctatgatgactacactag

SEQ ID NO: 3 - DNA, 33 bp, Artificial Sequence
  Forward elongation primer
acactctttccctacacgacgctcttccgatct

SEQ ID NO: 4 - DNA, 34 bp, Artificial Sequence
  Reverse amine primer
gtgactggagttcagacgtgtgctcttccgatct

SEQ ID NO: 5 - DNA, 57 bp, Artificial Sequence
  P5 adaptor primer
aatgatacggcgaaccaccgagatctacaaatagaggcacactctttccctacacga

SEQ ID NO: 6 - DNA, 57 bp, Artificial Sequence
  P7 adaptor primer
caagcagaagacggcatacgagatctgaagctgtgactggagttcagacgtgtgctc

SEQ ID NO: 7 - DNA, 9 bp, Artificial Sequence
  i5 index
aatagaggc

SEQ ID NO: 8 - DNA, 8 bp, Artificial Sequence
  i7 index
ctgaagct